(19) United States
(12) Patent Application Publication
(10) Pub. No.: US 2025/0064651 A1
(43) Pub. Date: Feb. 27, 2025

(54) ABSORBENT CORE

(71) Applicant: PRINCIPLE BUSINESS ENTERPRISES, INC., Bowling Green, OH (US)

(72) Inventor: Andrew J. Szypka, Curtice, OH (US)

(21) Appl. No.: 18/813,533

(22) Filed: Aug. 23, 2024

Related U.S. Application Data
(60) Provisional application No. 63/578,176, filed on Aug. 23, 2023.

(51) Int. Cl. A61F 13/53

(52) U.S. Cl. CPC A61F 13/53; A61F 2013/530481

(57) ABSTRACT

An absorbent article designed for improved fluid management is provided. The absorbent article features a multi-layered absorbent core comprising an acquisition layer, a surge layer, primary and secondary cores, and an optional storage layer. Each layer can be configured to optimize fluid intake, distribution, and retention, enhancing the overall performance of the article. The surge layer includes a super absorbent polymer with specific absorption rates and capacities, while the primary and secondary cores are designed with embossed patterns and voids to facilitate fluid movement and increase absorption efficiency. The absorbent core design reduces leakage, minimizes re-wetting, and decreases bulkiness, thereby improving wearer comfort and discretion. This technology is applicable to various types of absorbent articles, including diapers, training pants, and incontinence pads, addressing the needs of diverse user groups.

### CROSS-REFERENCE TO RELATED APPLICATIONS

This application claims the benefit of U.S. Provisional Application No. 63/578,176 filed on Aug. 23, 2023. The entire disclosure of the above application is incorporated herein by reference.

### FIELD

The present invention relates generally to an absorbent core and, in particular, to an absorbent core for a wearable article.

### INTRODUCTION

This section provides background information related to the present disclosure which is not necessarily prior art.

Absorbent articles such as diapers, incontinence pads, and training pants can be used for managing bodily fluids, providing comfort, and maintaining skin health. Certain absorbent articles utilize various materials and structures to capture and retain fluids. However, these designs often struggle with issues such as leakage, poor fluid distribution, and uncomfortable bulkiness. These problems primarily arise from the limitations in the materials used and the overall design of an absorbent core incorporated in the absorbent article.

One challenge for an absorbent article is the intake rate and effective distribution of fluid. Certain products use a single-layer absorbent core that may quickly become saturated, leading to leakage and discomfort. This is particularly problematic in scenarios where a high volume of fluid is expelled in a short period. Moreover, the inability to distribute fluid evenly throughout the absorbent core can result in inefficient use of the absorbent material, limiting effectiveness and further exacerbating leakage problems.

Another issue faced by an absorbent article relates to fluid retention. While some materials may absorb fluids quickly, certain materials do not necessarily lock the moisture away from the skin, which can lead to skin irritation and discomfort. Additionally, the re-wetting phenomenon, where fluids move back to the surface of the absorbent article, remains a significant concern, as it can compromise skin health and wearer comfort.

Bulkiness of absorbent articles is another issue. High-absorbency articles can be thick and cumbersome, making them uncomfortable and conspicuous under clothing. This bulkiness can restrict the wearer's movement and lead to a negative impact on the user's quality of life, particularly for active individuals or those desiring discretion about their absorbent product use.

Accordingly, there is a need to provide an improved absorbent core for an absorbent article that can better absorb and retain fluid and other discharge deposited onto the article while providing improved skin health. Desirably, the absorbent article should feature a multi-layered absorbent core that can manage fluids more efficiently, ensure rapid absorption, effective fluid distribution, and minimal re-wetting. Additionally, the absorbent core should minimize the bulkiness of the absorbent article yet still provide high absorbency, thus enhancing wearer comfort and discretion.

### SUMMARY

In concordance with the instant disclosure, an improved absorbent core for an absorbent article has surprisingly been discovered. The present technology includes articles of manufacture, systems, and processes that relate to advanced absorbent articles configured for enhanced fluid management, improved wearer comfort, and increased efficiency in fluid absorption and retention.

In certain embodiments, an absorbent core configured for use in an absorbent article is provided. The absorbent core can include a combination of several distinct layers: an acquisition layer, a surge layer, a primary core, a secondary core, and a storage layer. Each of these layers can be configured with specific materials and properties to enhance the overall functionality of the absorbent article. The acquisition layer can be made from non-woven polyester or polyolefin materials, providing initial fluid capture and transfer capabilities. The surge layer can include a high permeability super absorbent polymer, which is essential for managing one or more significant fluid surges and distributing such fluid surges evenly across the core.

The primary core can include a mix of super absorbent polymer and fluff, with the super absorbent polymer proportion specifically tailored to optimize fluid absorption and retention. The primary core can also be embossed with a straight pattern and can include a primary elongate void to facilitate rapid fluid absorption and prevent leakage. The secondary core, similarly, can be composed of cellulosic pulp fluff and super absorbent polymer, with the super absorbent polymer making up a significant percentage of the core's composition. The secondary core layer can be embossed with wavy lines to enhance structural integrity and fluid management.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic view of an absorbent core for an absorbent article, according to one embodiment of the present disclosure.

FIG. 2 is a cross sectional, side elevational view through the absorbent core taken at section line 2 in FIG. 1.

FIG. 3 is a top perspective view of a primary core of the absorbent core of FIG. 1.

FIG. 4 is a top perspective view of a secondary core of the absorbent core of FIG. 1.

FIG. 5 is a cross sectional, side elevational view through the primary core taken at section line 5 in FIG. 4.

FIG. 6 is a cross sectional, side elevational view through the secondary core taken at section line 6 in FIG. 1.

FIG. 7 is a top plan view of an absorbent article with the absorbent core.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology improves upon existing absorbent article designs by incorporating a multi-layered absorbent core that optimizes fluid intake, distribution, and retention. This innovative approach reduces the risk of leakage and enhances the dryness felt by the wearer. Additionally, the technology integrates materials that are both highly absorbent and less bulky, providing a more comfortable and discreet solution for users. These advancements collectively address the common issues faced by absorbent articles and offer significant improvements in both functionality and wearer satisfaction.

Throughout this detailed description, reference is made to top/upper and bottom/lower absorbent cores or layers of an absorbent core. It should be understood that, in general, the inner core or layer refers to a core or layer closer to the body or skin of the user as compared to a lower core or layer, where the lower core or layer refers to a core or layer further from the body or skin of the user as compared to an upper core or layer. However, unless expressly indicated, this convention is only to aid in referencing the various layers and is not intended to limit the scope of the disclosed or claimed embodiments.

Absorbent articles are provided that include a moisture-pervious inner layer, an absorbent core, and a moisture-impervious outer layer. Although the present detailed description relates the absorbent article to an incontinence article, such as a disposable diaper, it is to be understood that embodiments of the absorbent article including the absorbent core can also be implemented into other absorbent articles and that the properties and uses described below apply to these other absorbent articles as well. Throughout this application, the terms absorbent article and diaper are used interchangeably. However, it should be understood that the term diaper is intended to include other absorbent articles, such as training pants, incontinence pads, etc., as would be understood by one of ordinary skill in the art.

In certain embodiments, the absorbent article can include multiple layers, including a top layer, an absorbent core, and a bottom layer. The top layer faces a wearer and contacts the skin of the wearer when the absorbent article 100 is secured to the wearer. The top layer can include a topsheet having a moisture-pervious fabric suitable to allow bodily discharge to pass through the top layer and be absorbed by the absorbent core. Example materials for the topsheet include polypropylene, polyethylene, polyester, materials having hydrophobic properties, combinations thereof and/or the like. Additionally, the topsheet can be treated with a hydrophilic coating or finish to improve passthrough of liquids to diaper layers beneath the inner layer. Non-limiting examples of suitable hydrophilic coatings include stearic acid, melamine-based coatings, fluorocarbons, and silicon-based coatings.

In certain embodiments, the absorbent core can include an acquisition layer, a surge layer, a primary core, a secondary core, and a storage layer. It should be appreciated that the absorbent core can include various combinations of layers as described herein. For example, the absorbent core does not necessarily need to include the storage layer for all applications. A skilled artisan can select and combine the layers, as described herein, based on the needs for a particular user of a particular absorbent article to be formed.

The acquisition layer can capture fluids upon contact and efficiently transfer them to one or more lower layers. The acquisition layer can be configured to reduce a sensation of wetness for the wearer and militate against a fluid resurfacing, which enhances comfort and promotes skin health. The acquisition layer can be configured to receive fluid, in operation. The acquisition layer can be configured to receive the fluid without wicking or spreading the fluid throughout the layer. In certain embodiments, the acquisition layer can include a non-woven polyester material, other polyolefin materials, and combinations thereof. In a particular non-limiting example, the acquisition layer can include a resin bonded at 40 grams per square meter (gsm) non-woven polyester.

Positioned after the acquisition layer, the surge layer can manage fluid intake during instances of high fluid discharge. The surge layer can be configured to receive fluid from the acquisition layer and can absorb and distribute the fluid across the surge layer. The surge layer can act as a temporary reservoir, holding the fluid momentarily to prevent overflow and allowing more time for the subsequent layers to absorb the fluid effectively.

In certain embodiments, the surge layer can include a GELOCK® material; commercially available from Gelok International in Dunbridge, Ohio. In a particular non-limiting example, the material can be a low basis weight, air laid, hydrogen bonded GELOCK® material. The surge layer can include super absorbent polymer. The super absorbent polymer can be a high permeability super absorbent polymer. In some embodiments, the super absorbent polymer of the surge layer can have a permeability of 0.1×10⁻⁷ cm sec/g to 0.5×10⁻⁷ cm sec/g, and more particularly 0.2×10⁻⁷ cm sec/g. The super absorbent polymer can have a slow absorption speed. In some embodiments, the super absorbent polymer of the surge layer 106 can have an absorption rate of 40 seconds to 60 seconds, and more particularly 51 seconds. A non-limiting example of an acceptable super absorbent polymer for the surge layer can be AQUA KEEP HP650, commercially available from SUMITOMO SEIKA CHEMICALS CO., LTD. with offices in Osaka, Japan.

The super absorbent polymer of the surge layer can be present in a relatively small amount of a total amount of super absorbent polymer of the absorbent core. For example, the super absorbent polymer can be present in the surge layer in an amount of 0 to 50 gsm, and more particularly, 20-40 gsm, which can represent less than 5% of the total super absorbent polymer of the absorbent core, and more particularly, less than 2% of the total super absorbent polymer of the absorbent core.

In certain embodiments, the surge layer can be embossed. The surge layer can be embossed with a pattern configured to move fluids through and across the surge layer, in operation. The pattern can also improve an overall softness of the surge layer, which is beneficial due to the proximity of the surge layer to the user, in operation. In one non-limiting example, the embossed pattern can include wavy lines embossed along a length of the surge layer.

The primary core can serve as a main absorbent component of the absorbent article. The primary core can be configured to restrict lateral movement of fluids, promoting vertical absorption and militating against leakage, which is important for maintaining the integrity of the absorbent article. The primary core can be configured to receive fluid from the surge layer 106 and can absorb fluid while mitigating lateral movement of the fluid across the primary core 108. In certain embodiments, the primary core can be fabricated from a fluff material as well as a super absorbent polymer. In certain embodiments, the super absorbent polymer can be present in an amount of 15-20% of the primary core, while the fluff makes up approximately 80-85% of the material of the primary core. In certain other embodiments, the super absorbent polymer can be present in an amount of 50% or more of the primary core, while the fluff makes up approximately 50% or less of the material of the primary core.

The super absorbent polymer can be a high permeability super absorbent polymer. In some embodiments, the super absorbent polymer of the primary core can have a permeability of 0.1×10⁻⁷ cm sec/g to 0.5×10⁻⁷ cm sec/g, and more particularly 0.2×10⁻⁷ cm sec/g. The super absorbent polymer can have a fast absorption rate. In some embodiments, the super absorbent polymer of the primary core can have an absorption rate of 30 seconds to 60 seconds, and more particularly 42 seconds. A non-limiting example of an acceptable super absorbent polymer for the surge layer includes AQUA KEEP SA60SXII, commercially available from SUMITOMO SEIKA CHEMICALS CO., LTD. with offices in Osaka, Japan.

The super absorbent polymer of the primary core can be present in a relatively large amount of a total amount of super absorbent polymer of the absorbent core. For example, the super absorbent polymer can be present in the primary core in an amount of 30-50% of the total super absorbent polymer of the absorbent core, and more particularly, 40% of the total super absorbent polymer of the absorbent core. The super absorbent polymer of the primary core can be specifically applied within a central width of the core, aligning with the width of a crotch area of certain absorbent articles. The application of the super absorbent polymer of the primary core allows the super absorbent polymer to extend a full length of the core but confined to the central region where fluid is most likely to be deposited during use. Embodiments further include where he super absorbent polymer is intentionally not included in the extended width of the primary core at the front and back of the core, as fluid typically does not migrate to these peripheral areas. By concentrating the super absorbent polymer in the central area where fluid accumulation is most common, the absorbent article can use super absorbent polymer more efficiently, enhancing absorption where it is needed most while conserving material where it is less likely to contact fluid.

In certain embodiments, the primary core can be embossed. The primary core 108 can be embossed with a pattern configured to densify the material of the primary core 108. Densify in this context means to make the material more compact. This increased density can aid in controlling the movement and absorption of fluids within the core, making it more efficient at absorbing and retaining fluids without spreading them laterally. In one non-limiting example, the embossed pattern can include a straight pattern embossed along a length and a width of primary core.

In certain embodiments, the primary core can include one or more primary elongate voids formed therethrough. The primary elongate void can be a cut-out, cavity, opening, or similar feature formed through a height of the primary core. The primary elongate void can be configured to increase the absorption rate of fluid through the primary core by allowing for a direct path for a liquid to spread relatively unimpeded through the primary core. A skilled artisan can select appropriate shapes and dimensions for the primary elongate void, as required for the absorbent core. More than one elongate void and type of elongate void can also be implemented by the skilled artisan, as desired.

Located below the primary core, the secondary core can provide additional absorbency and retention capacity. The secondary core can absorb any excess fluid that passes through the primary core, ensuring that the absorbent article remains effective over extended periods. The secondary core can be configured to receive fluid from the primary core. In certain embodiments, the secondary core can include a fluff material (e.g., a cellulosic pulp fluff) as well as a super absorbent polymer. In certain embodiments, the super absorbent polymer can be present in an amount of 50% of the secondary core, while the fluff can make up approximately 50% of the material of the secondary core. In other embodiments, the super absorbent polymer can make up more than 50% of the secondary core.

The super absorbent polymer of the secondary core can provide fast absorption, high capacity, and high retention of a liquid. It should be appreciated that these relative performance indicators of the absorbent polymer of the secondary core can be measured according to the Edana Nonwovens Standard Procedures by a person having skill in the art. For example, high retention means that the super absorbent polymer can retain the absorbed liquid under a subsequent application of a pressure, but also that the superabsorbent polymer be capable of absorbing liquids even against a simultaneously exerted pressure (e.g., applied during the liquid absorption process) of the kind encountered in practice when an infant or adult sits or lies on a sanitary article or when shearing forces are developed, for example as a result of motion of the legs. This specific absorption characteristic is referred to in Edana Nonwovens Standard Procedures as “Absorbency Against Pressure” or AAP for short. The AAP value reported for a superabsorbent polymer is based on the pressure employed, for example, 21 g/cm² at 0.3 psi and 50 g/cm² at 0.7 psi. Also, the AAP may be based on the ratio chosen for the measurement of the superabsorbent polymer weight to area, for example 0.032 g per cm², and also by the particle size distribution of a granular superabsorbent polymer.

The super absorbent polymer of the secondary core can be present in a relatively large amount of a total amount of super absorbent polymer of the absorbent core. For example, the super absorbent polymer can be present in the secondary core in an amount of 50-70% of the total super absorbent polymer of the absorbent core, and more particularly, 60% of the total super absorbent polymer of the absorbent core.

In certain embodiments, the secondary core can be embossed. The secondary core 110 can be embossed with a pattern configured to densify the material of the primary core 108. In one non-limiting example, the embossed pattern can include wavy lines embossed along a length of the secondary core.

The storage layer, situated as a barrier within the absorbent core, can securely hold excess fluid. The storage layer can militate against leaks and ensure that the outer layers of the absorbent article stay dry, providing comfort and security to the wearer and caregivers. The storage layer can be configured to receive excess fluid and absorb excess fluid from the secondary core. In certain embodiments, the storage layer can include super absorbent, cellulose paper laminate. In certain embodiments, the super absorbent polymer can be 14040-72 laminate GELOCK® material (commercially available from Gelok International in Dunbridge, Ohio). The storage layer can also include super absorbent polymer. A non-limiting example of an acceptable super absorbent polymer for the surge layer can be AQUA KEEP SA60SXII, commercially available from SUMITOMO SEIKA CHEMICALS CO., LTD. with offices in Osaka, Japan. The storage layer can include the super absorbent polymer.

It should be appreciated that in the absorbent core, where super absorbent polymer and fluff pulp fibers are used together, the super absorbent polymer and fluff pulp fibers can be homogeneously mixed to create a highly effective absorbent medium. A homogeneous mixture ensures that the super absorbent polymer, with its high fluid absorption capacity, can be evenly distributed throughout the fluff pulp, which serves as a bulking and distribution agent. The fluff pulp, typically made from cellulose fibers, can provide structural integrity to the absorbent core and facilitate the rapid wicking and dispersion of fluids across the absorbent core. When combined homogeneously with the super absorbent polymer, the fluff pulp can stabilize the super absorbent polymer particles, militating against the particles from clumping when wet and allowing for a more consistent absorption profile throughout the absorbent core. The cooperation between fluff pulp and super absorbent polymer not only increases fluid absorption but also maintains the structural stability of the absorbent core, militating against sagging or bunching that can lead to discomfort and leaks for the user.

As described herein, the absorbent core can incorporates strategic embossing patterns on multiple layers to enhance fluid management and structural integrity. Specifically, the surge layer, primary core, and secondary core are embossed with distinct patterns, each serving a unique purpose in optimizing the performance of the absorbent core.

The primary core can feature straight lines embossed along both its length and width. This embossing configuration is particularly beneficial as it helps to densify the material, which in turn aids in controlling fluid movement and absorption within the core. The straight line pattern is strategically designed to restrict lateral fluid movement while promoting vertical absorption, thereby reducing the risk of leakage and improving the overall performance of the absorbent article. The straight lines create a more compact structure, which contributes to the ability of the primary core to maintain its shape and effectiveness, even when saturated with fluid. By creating a more densified and compact material, the embossing helps the primary core maintain its shape and effectiveness over time.

The secondary core can be embossed with wavy lines along its length, similar to the surge layer. This wavy pattern likely serves to enhance fluid distribution and promote efficient absorption across the layer. The undulating lines can create channels that guide fluid flow, ensuring more uniform coverage and improving the ability of the secondary core to manage and retain fluids effectively.

The embossing process for these layers can be achieved during manufacturing using specialized embossing rollers or plates. For both the surge layer and secondary core, rollers with wavy patterns engraved around their circumference can be used to create the desired wavy line embossing. For the primary core, rollers with parallel grooves running along their length can be employed to create the straight line embossing. During the embossing process, the material passes between the patterned roller (or plate) and a smooth backing roller. Carefully controlled pressure is applied to imprint the desired pattern onto the material. In some cases, heat may also be applied to enhance the permanence of the pattern or to affect the material's properties. The pressure and temperature used during this process are meticulously regulated to achieve the optimal level of densification and pattern definition without compromising the absorbent properties of the materials. The strategic use of embossing patterns, combined with the manufacturing process, contributes to the overall effectiveness of the absorbent core. By enhancing fluid distribution, promoting targeted absorption, and maintaining structural integrity, these embossed layers work in concert to improve the article's performance in managing fluid absorption and retention, ultimately reducing the risk of leakage and enhancing user comfort.

As detailed herein, it should be appreciated that the absorbent core can include a variety of combinations of the layers described herein. For example, the absorbent core can include the storage layer for an absorbent article configured for long term use. It may not be necessary to include the storage layer for all applications. In other embodiments, the absorbent core can include the acquisition layer, the primary core, and the secondary core. However, it should be appreciated that the absorbent core can be arranged by a skilled artisan based on the needs for the absorbent article.

Advantageously, the present disclosure introduces a multi-layered absorbent core that enhances fluid management in absorbent articles. The absorbent core militates against leakage, reduce re-wetting, and distribute fluids evenly, ensuring that the absorbent core utilizes the full capacity more efficiently. Furthermore, the use of embossing techniques and advanced material compositions in the cores minimizes the overall bulkiness of the absorbent article, thereby providing a more comfortable and discreet solution for users.

### EXAMPLES

Example embodiments of the present technology are provided with reference to the several figures enclosed herewith.

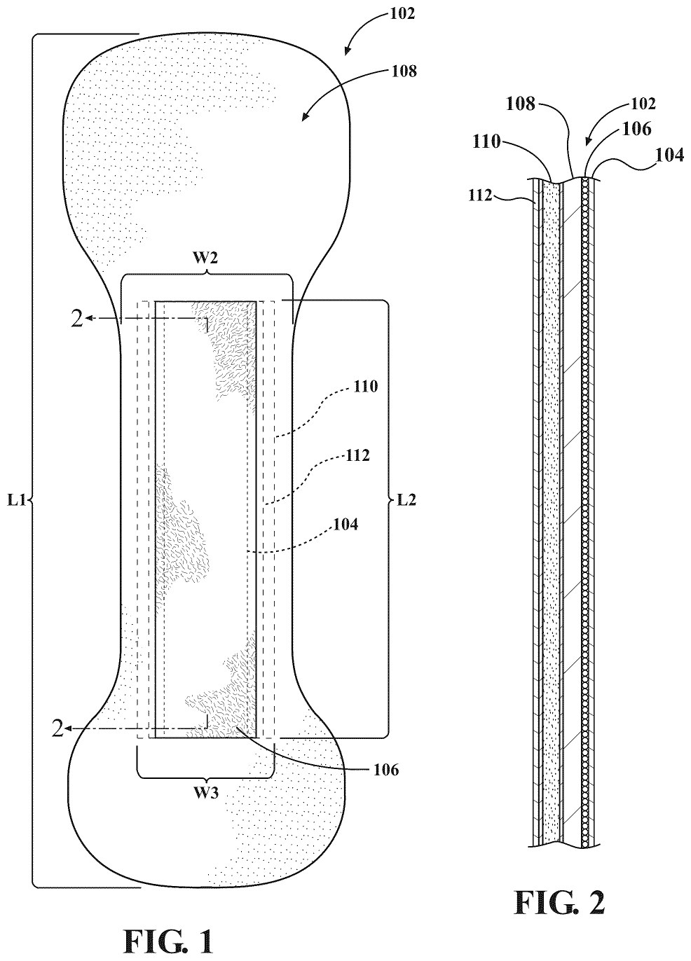

With reference to FIGS. 1-2, a first embodiment of an absorbent core 102 for an absorbent article 100 is shown. FIG. 2 provides a detailed cross-sectional view of the multi-layered absorbent core 100. The acquisition layer 104 is crafted from non-woven polyester, designed to capture and channel fluids to the layer below. Directly beneath, the surge layer 106 contains high permeability super absorbent polymer (SAP) and features embossed wavy lines to ensure even fluid distribution across the core. Following this, the primary core 108 includes a blend of fluff material and SAP, detailed with a straight line embossing pattern to restrict lateral fluid movement and promote uniform saturation. The secondary core 110, situated below the primary core, is a mix of fluff and high-capacity SAP, tasked with absorbing and retaining any excess fluid that traverses the primary core. The final layer, the storage layer 112, is designed to hold excess fluid and prevent saturation, ensuring the absorbent core remains effective over extended periods.

As depicted in FIG. 1, the primary core 108 can have a length (L1) greater than a length (L2) of the remainder of the absorbent core 100. The primary core 108 can include an elongate portion 114 that is configured to be disposed along a crotch portion 103 of an absorbent article 101. The elongate portion 114 can include wings 116 extending therefrom.

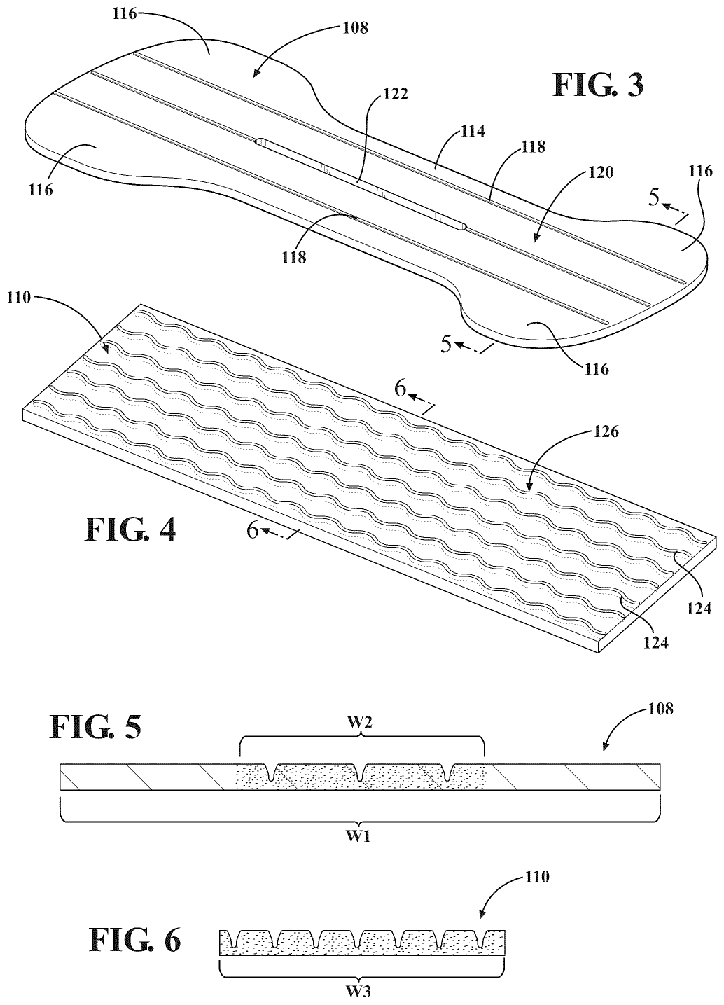

FIG. 3 depicts the primary core 108 including the straight embossing 118 formed on an underside 120 of the primary core. In particular, the embossing 118 includes three embossed lines disposed along the length (L1) of the primary core 108. A primary elongate void 122 can be a cut-out, cavity, opening, or similar feature formed through a height of the primary core 108 and a long a portion of the length (L1). The primary elongate void 122 can be configured to increase the absorption rate of fluid through the primary core by allowing for a direct path through the primary core 108.

As depicted in FIGS. 3 and 5, the elongate portion 114 can have a width (W1) and the wings 116 can have a width (W2). As discussed in greater detail herein, the wings 116 do not include the super absorbent polymer. All of the super absorbent polymer of the primary core 108 can be disposed in the width (W2) of the elongate portion 114 of the primary core 108. The embossing 118 of the primary core 108 can also be disposed on the elongate portion 114 and the width (W2) and not in the wings 116. The width (W2) of the primary core 108 can be wider than a width (W3) of the secondary core 110, for example as shown in FIGS. 1 and 4-5. The width (W2) of the elongate portion 114 can be substantially the same as the width (W3) of the secondary core 110.

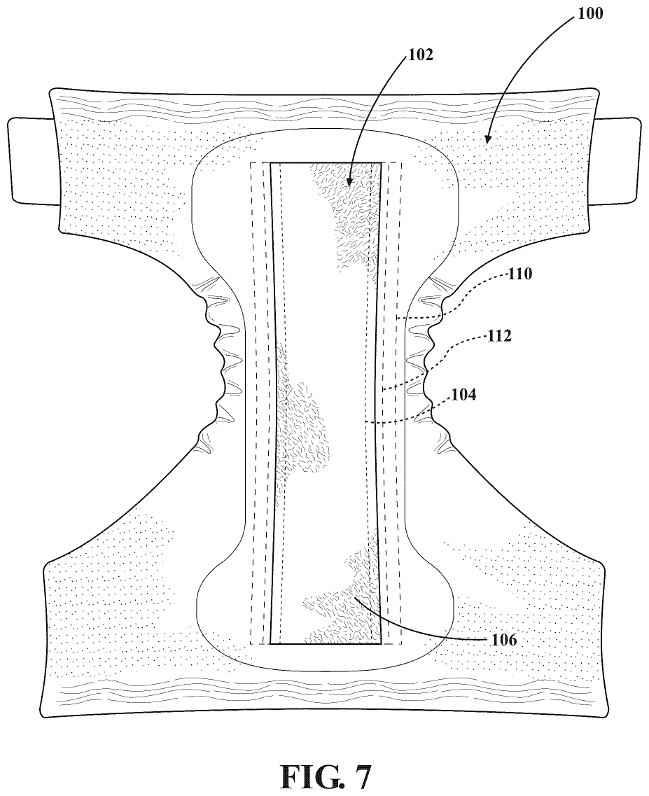

FIGS. 4 and 6 depict the secondary core 110 and the wavy line embossing 124 formed thereon. In particular, the embossing 124 can be formed in a bottom side 126 of the secondary core 110. The embossing 124 can be formed across the entire width (W3) and the entire length (L3) of the secondary core. FIG. 7 depicts an absorbent article 100 including the absorbent core 102 of FIG. 1. The core 102 can be arranged substantially centrally in a crotch portion of the absorbent article 100.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. An absorbent core for an absorbent article, comprising:
an acquisition layer;
a surge layer;
a primary core; and
a secondary core.

2. The absorbent core of claim 1, wherein the acquisition layer includes a member selected from a group consisting of a non-woven polyester, a non-woven polyolefin material, and combinations thereof.

3. The absorbent core of claim 1, wherein the surge layer includes a super absorbent polymer that is a high permeability super absorbent polymer.

4. The absorbent core of claim 1, wherein the surge layer includes 20-40 gsm of super absorbent polymer.

5. The absorbent core of claim 1, wherein the primary core includes super absorbent polymer and fluff, and the super absorbent polymer is present in an amount of 15-20% of the primary core.

6. The absorbent core of claim 1, wherein the primary core includes a high permeability super absorbent polymer.

7. The absorbent core of claim 1, wherein the primary core includes no more than 50% of the total super absorbent polymer of the absorbent core.

8. The absorbent core of claim 1, wherein the primary core is embossed with a straight line pattern covering a length and a width of the primary core.

9. The absorbent core of claim 1, wherein the primary core includes a primary elongate void formed therethrough.

10. The absorbent core of claim 1, wherein the secondary core is fabricated from a cellulosic pulp fluff material and a super absorbent polymer, and the super absorbent polymer comprises at least 50% of the secondary core.

11. The absorbent core of claim 1, wherein the secondary core includes a fast absorption, high capacity, and high retention super absorbent polymer.

12. The absorbent core of claim 1, wherein the super absorbent polymer present in the secondary core comprises at least 50% of the total super absorbent polymer of the absorbent core.

13. The absorbent core of claim 1, wherein the secondary core is embossed with wavy lines along a length of the secondary core.

14. The absorbent core of claim 1, wherein the storage layer includes a cellulose paper laminate.

15. An absorbent article comprising the absorbent core of claim 1.

16. The absorbent core of claim 15, further comprising a storage layer.

17. The core of claim 1, wherein the primary core is embossed with a straight line pattern covering a length and a width of the primary core and the secondary core is embossed with wavy lines along a length of the secondary core.

18. The core of claim 1, wherein the primary core can include an elongate portion and wings extending therefrom.

19. The core of claim 18, wherein the elongate portion has a width and the secondary core has a width and the width of the elongate portion is substantially the same as the width of the secondary core.

20. An absorbent core for an absorbent article, comprising:
an acquisition layer fabricated from a non-woven polyester or polyolefin material and combinations thereof;
a surge layer that includes 20-40 gsm of a high permeability super absorbent polymer and is embossed with wavy lines along its length;
a primary core that includes less than 50% of the total super absorbent polymer of the absorbent core, with the super absorbent polymer present in an amount of less than 50% of the primary core and mixed with fluff, further embossed with a straight line pattern along both the length and the width, and includes a primary elongate void formed therethrough;
a secondary core fabricated from a cellulosic pulp fluff material and a super absorbent polymer, with the super absorbent polymer present in an amount of at least 50%, and embossed with wavy lines along its length, designed for fast absorption, high capacity, and high retention; and
a storage layer fabricated from cellulose paper laminate.
